(19) United States
(12) Patent Application Publication
(10) Pub. No.: US 2021/0030999 A1
(43) Pub. Date: Feb. 4, 2021

(54) DEVICES, SYSTEMS, AND METHODS FOR USER CALIBRATION OF MECHANICAL STIMULATION PARAMETERS

(71) Applicant: Apex Neuro Holdings, Inc., Cambridge, MA (US)

(72) Inventor: Francois Kress, New York, NY (US)

(21) Appl. No.: 16/943,204

(22) Filed: Jul. 30, 2020

Related U.S. Application Data
(60) Provisional application No. 62/881,700, filed on Aug. 1, 2019.

(51) Int. Cl. A61M 21/02; A61H 23/02

(52) U.S. Cl. CPC A61M 21/02; A61M 2021/0022; A61H 23/02

(57) ABSTRACT

Systems, methods, and devices that provide mechanical stimulation to a user through a stimulation device. In one embodiment, a stimulation device containing a plurality of mechanical waves to a user, each mechanical wave having one or more vibrational waveforms is provided. More than one of the plurality of mechanical waves are delivered to the user to a location in proximity to a mastoid of the user. The processor of the stimulation device or a computing device in communication with the stimulation device receives the user's selection of or preference for of mechanical waves that the user prefers. The mechanical waves provided to the user are modified or another mechanical waves are selected to provide to the user based at least in part on the user's selection of or preference for one or more of the plurality of mechanical waves which is delivered to the user.

### CROSS REFERENCE TO RELATED APPLICATIONS

This application claims the benefit of and priority to U.S. Provisional Application No. 62/881,700, entitled “DEVICES, SYSTEMS, AND METHODS FOR USER CALIBRATION OF MECHANICAL STIMULATION PARAMETERS”, filed on Aug. 1, 2019, and the earlier filing date of U.S. Provisional Application No. 62/881,700, the teachings and entire disclosure of which are hereby expressly incorporated herein by reference in their entirety for all purposes.

### FIELD OF THE INVENTION

The present invention relates generally to wearable mechanical stimulation and neuromodulation devices for promoting nerve stimulation through mechanical vibration and related methods and systems.

### BACKGROUND

Electrical stimulation of nerves in human subjects can alter mood states, reduce the sensation of pain, and treat certain diseases. While promising in this regard, patients subjected to electrical stimulation often experience unpleasant and/or dangerous side effects, including but not limited to skin irritation resulting from gels needed to maintain good contact between electrodes and the patient's skin, burns and/or rashes, and pain or irritation at the stimulation site. Such side effects are particularly problematic for applications where nerve stimulation should be applied frequently (e.g., daily), such as for stress management. Additionally, subjects having a negative experience or experiencing the side effects mentioned above are likely to not optimize or, even work, continue to their treatments due to the negative experiences and/or side effects. The present disclosure demonstrates that when the proper parameters of effective signals is combined with customization of the signal and treatment by the patient, a synergy is created that dramatically improves the result to the patient both because the treatment is more effective and because the patient, having customized the treatment is more likely to complete and obtain the full benefits from the device, system or method of treatments.

Accordingly, there is a need for systems, methods, and devices that provide for convenient, regular nerve stimulation with limited side effects and a robust safety profile. Such systems, methods, and devices are of particular relevance to the treatment of conditions where frequent nerve stimulation is desired.

### SUMMARY OF THE INVENTION

Presented herein are systems, methods, and devices that provide for convenient and intuitive user-calibration of delivery parameters (e.g., frequency, amplitude, duration, etc.) that set characteristics of mechanical stimulation provided to a user via a stimulation device. The approaches described herein allow a user to, among other things, refine and personalize the specific stimulation that they receive, based on their personal preferences and what provides the best results and outcome and thus is best suited based on their unique physical traits and condition(s).

In one aspect, the disclosure is directed to a method of calibrating values of one or more delivery parameters (e.g., a frequency, amplitude, duration, etc.) of transcutaneous mechanical stimulation provided to a user (e.g., a subject) via a stimulation device, the method comprising: (a) generating (e.g., by the stimulation device)(e.g., sequentially) a plurality of test mechanical waves, each having a vibrational waveform based at least in part on a corresponding test set of values for the one or more delivery parameters; delivering (e.g., by the stimulation device)(e.g., sequentially) each of the test mechanical waves to the user [e.g., at a body location (e.g., a location in proximity to a mastoid of the user)]; (c) receiving [e.g., by a processor of a user computing device (e.g., a mobile phone; e.g., a tablet computer; e.g., a personal computer)(e.g., in communication with, the stimulation device)(e.g., via a graphical user interface of the user computing device); e.g., by a processor of the stimulation device (e.g., via a user interaction with one or more control elements, such as buttons, touch sensitive surfaces, etc., of the stimulation device)] a user selection corresponding to an indication of preference for one or more of the test mechanical waves; (d) determining (e.g., by the processor of the user computing device; e.g., by the processor of the stimulation device) a calibrated set of values for the one or more delivery parameters based at least in part on the user selection; (e) generating (e.g., by the stimulation device) a calibrated mechanical wave having a vibrational waveform based at least in part on the calibrated set of values for the one or more delivery parameters; and (f) delivering (e.g., by the stimulation device) the calibrated mechanical wave to the user [e.g., at the body location (e.g., a location in proximity to a mastoid of the user)], thereby providing user calibrated transcutaneous mechanical stimulation to the user.

In other embodiments, the method comprises iteratively performing steps (a)-(c) while updating (e.g., by the processor of the user computing device and/or by the processor of the stimulation device) the delivery parameter values in one or more of the test sets based on the user selection (e.g., at set times, after periods of continuous use, e.g., every day, 7 days, 30 days, etc.) to refine values of the delivery parameters over a set period of time (e.g., during an initial calibration phase) and/or until a particular stop criteria (e.g., a user input provided via the stimulation device and/or the user computing device; e.g., convergence of the values of the one or more delivery parameters) is reached, thereby determining the calibrated set of values.

In yet other embodiments, step (b) comprises, for each of the mechanical waves, providing, to the user [e.g., via the stimulation device; e.g., via a user computing device (e.g., in communication with the stimulation device)], an alert (e.g., via the graphical user interface (e.g., of an app) of the user computing device; e.g., via haptic, audio, visual, or other feedback provided through the stimulation device and/or the user computing device); indicating delivery of each mechanical wave.

In yet other embodiments, the one or more delivery parameters comprise one or more members selected from the group consisting of a frequency (e.g., of the vibrational waveform), an amplitude (e.g., of the vibrational waveform), and a duration (e.g., of the mechanical stimulation).

In yet other embodiments, the method comprises receiving (e.g., from a wearable monitoring device; e.g., from one or more sensors of the stimulation device)(e.g., a fitness tracker (e.g., pedometer); e.g., a heart-rate monitor; e.g., an electrocardiograph (EKG) monitor; e.g., an electroencephalography (EEG) monitor; e.g., an accelerometer; e.g., a blood-pressure monitor; e.g., a galvanic skin response (GSR) monitor); one or more biofeedback signals (e.g., step count, hear-rate statistics, such as average heart rate, heart-rate variability, etc., EKG, EEG, blood pressure, GSR, etc.); and determining and/or updating the calibrated set of values based on the received one or more biofeedback signals.

In certain embodiments, at least one of the corresponding test sets comprises values selected to produce a vibrational waveform for treating anxiety and/or a related disorder (e.g., to promote stimulation of a vagal nerve of the user; e.g., to promote stimulation of C-tactile afferents (e.g., comprising values of parameters, such as frequencies, selected to be at extremes of C-tactile afferent response domains); e.g., to promote stimulation of Piezo2 proteins).

In yet other embodiments, at least one of the corresponding test sets comprises values selected to produce a vibrational waveform that promotes stimulation of one or more members elected from the group consisting of: a vagal nerve of the user; a trigeminal nerve of the user; a C-tactile afferent; a Piezo2 protein, a Merkel cell.

In yet other aspects of at least some embodiments, the invention is directed to a transcutaneous neuromodulation device (a stimulation device) for providing calibrated transcutaneous mechanical stimulation to a user (e.g., a subject) device comprising: one or more mechanical transducers operable to produce mechanical waves in response to an electronic drive signal and positioned (e.g., via a housing and ergonomic support components of the transcutaneous neuromodulation device) to deliver the mechanical waves to the user at one or more body locations (e.g., in proximity to one or more mastoid locations); and a controller board in communication with the one or more mechanical transducers and with a processor of a computing device [e.g., a processor of the neuromodulation device (e.g., of the controller board); e.g., a processor of a user computing device], wherein the controller board is operable to (e.g., wherein the controller board comprises a controller board processor and controller board memory having instructions stored therein, wherein the instructions, when executed by the controller board processor, cause the processor to): control a waveform of the electronic drive signal to generate a plurality of test mechanical waves, each having a vibrational waveform based at least in part on a corresponding test set of values for the one or more delivery parameters; receive, via the computing device, a calibrated set of values for the one or more delivery parameters, said calibrated set of values determined by the computing device and based at least in part on a user selection corresponding to an indication of preference for one or more of the test mechanical waves; and control the waveform of the electronic drive signal to generate a calibrated mechanical wave having a vibrational waveform based at least in part on the calibrated set of values for the one or more delivery parameters, thereby providing calibrated transcutaneous mechanical stimulation to the user.

In another aspect, at least one embodiment of the present disclosure is directed to a system for calibrating values of one or more delivery parameters (e.g., a frequency, amplitude, duration, etc.) of transcutaneous mechanical stimulation provided to a subject via a stimulation device, the system comprising: a processor of a computing device (e.g., a user computing device; e.g., in communication with the stimulation device); and a memory having instructions stored thereon, wherein the instructions, when executed by the processor, cause the processor to: (a) cause generation and delivery (e.g., by the stimulation device)(e.g., sequentially) of a plurality of test mechanical waves, each having a vibrational waveform based at least in part on a corresponding test set of values for the one or more delivery parameters; (b) receive [e.g., by a processor of a user computing device (e.g., a mobile phone; e.g., a tablet computer; e.g., a personal computer)(e.g., in communication with, the stimulation device)(e.g., via a graphical user interface of the user computing device); e.g., by a processor of the stimulation device (e.g., via a user interaction with one or more control elements, such as buttons, touch sensitive surfaces, etc., of the stimulation device)] a user selection corresponding to an indication of preference for one or more of the test mechanical waves; (c) determine (e.g., by the processor of the user computing device; e.g., by the processor of the stimulation device) a calibrated set of values for the one or more delivery parameters based at least in part on the user selection; and (d) cause generation and delivery of (e.g., by the stimulation device) a calibrated mechanical wave having a vibrational waveform based at least in part on the calibrated set of values for the one or more delivery parameters, thereby providing user calibrated transcutaneous mechanical stimulation to the subject.

In at least some embodiments, the instructions cause the processor to iteratively perform steps (a)-(b) while updating the delivery parameter values in one or more of the test sets based on the user selection (e.g., at set times, after periods of continuous use, e.g., every day, 7 days, 30 days, etc.) to refine values of the delivery parameters over a set period of time (e.g., during an initial calibration phase) and/or until a particular stop criteria (e.g., a user input provided via the stimulation device and/or the user computing device; e.g., convergence of the values of the one or more delivery parameters) is reached, thereby determining the calibrated set of values.

In certain aspect of at least some embodiments, step (a) comprises, for each of the mechanical waves, providing, to the user [e.g., via the stimulation device; e.g., via a user computing device (e.g., in communication with the stimulation device)], an alert (e.g., via the graphical user interface (e.g., of an app) of the user computing device; e.g., via haptic, audio, visual, or other feedback provided through the stimulation device and/or the user computing device); indicating delivery of each mechanical wave.

In certain aspect of at least some embodiments, the one or more delivery parameters comprise one or more members selected from the group consisting of a frequency (e.g., of the vibrational waveform), an amplitude (e.g., of the vibrational waveform), and a duration (e.g., of the mechanical stimulation).

In certain aspect of at least some embodiments, the instructions cause the processor to: receive (e.g., from a wearable monitoring device; e.g., from one or more sensors of the stimulation device)(e.g., a fitness tracker (e.g., pedometer); e.g., a heart-rate monitor; e.g., an electrocardiograph (EKG) monitor; e.g., an electroencephalography (EEG) monitor; e.g., an accelerometer; e.g., a blood-pressure monitor; e.g., a galvanic skin response (GSR) monitor); one or more biofeedback signals (e.g., step count, hear-rate statistics, such as average heart rate, heart-rate variability, etc., EKG, EEG, blood pressure, GSR, etc.); and determine and/or update the calibrated set of values based on the received one or more biofeedback signals.

In certain aspects of at least some embodiments, at least one of the corresponding test sets comprises values selected to produce a vibrational waveform for treating anxiety and/or a related disorder (e.g., to promote stimulation of a vagal nerve of the user; e.g., to promote stimulation of C-tactile afferents (e.g., comprising values of parameters, such as frequencies, selected to be at extremes of C-tactile afferent response domains); e.g., to promote stimulation of Piezo2 proteins).

In certain aspect of at least some embodiments, at least one of the corresponding test sets comprises values selected to produce a vibrational waveform that promotes stimulation of one or more members elected from the group consisting of: a vagal nerve of the user; a trigeminal nerve of the user; a C-tactile afferent; a Piezo2 protein, a Merkel cell.

Some elements of at least some embodiments involving one aspect of the invention (e.g., compositions, e.g., systems, e.g., methods) can be applied in embodiments involving other aspects of the invention, and vice versa.

### BRIEF DESCRIPTION OF THE DRAWINGS

The foregoing and other objects, aspects, features, and advantages of the present disclosure will become more apparent and better understood by referring to the following description taken in conjunction with the accompanying drawings, in which:

FIGS. 1-23 are screenshots of a graphical user interface (GUI) of a mobile computing application that guide a user through a calibration process to personalize their mechanical stimulation, according to an illustrative embodiment. The GUI alerts the user to when different waves are being provided, and solicits feedback from the user of their preference for one wave over another.

FIGS. 24-47 are screenshots of an exemplary GUI of a mobile computing application for controlling a device for delivery of transcutaneous mechanical stimulation in accordance with the systems, methods, and devices described herein. The GUI includes the calibration guide shown in FIGS. 1-23, as well as other screens providing guidance and control in use of the device and adjusting various settings.

FIG. 48 is a block flow diagram of a process for calibrating delivery parameters of mechanical stimulation, according to an illustrative embodiment.

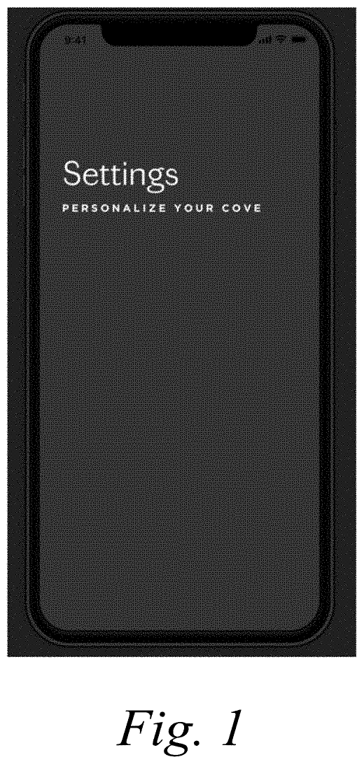

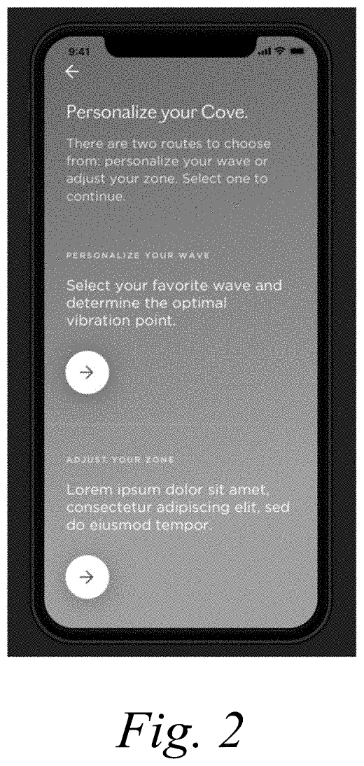

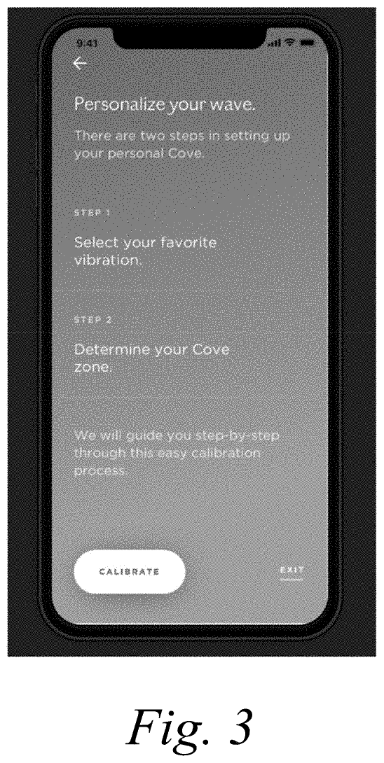

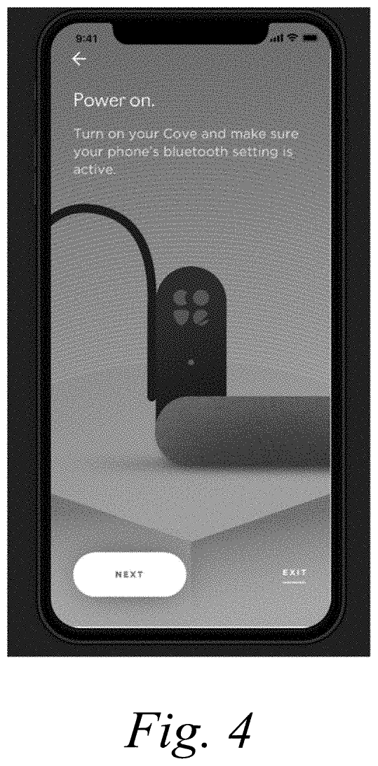

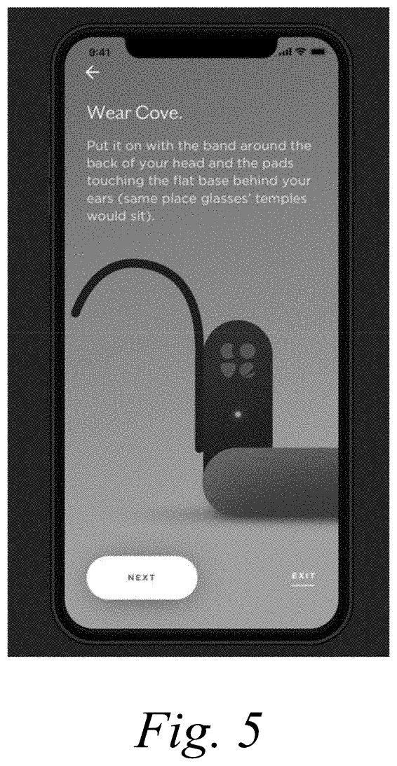

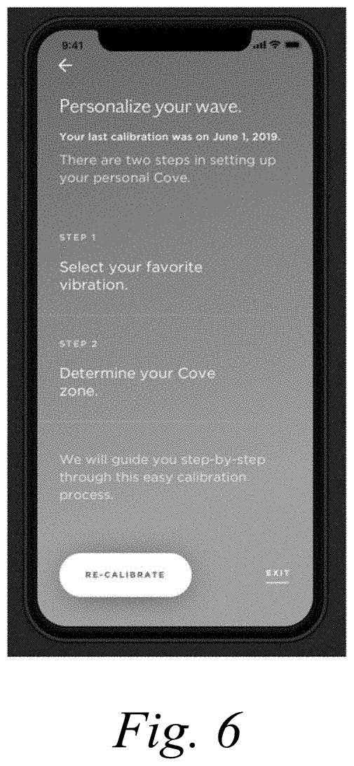

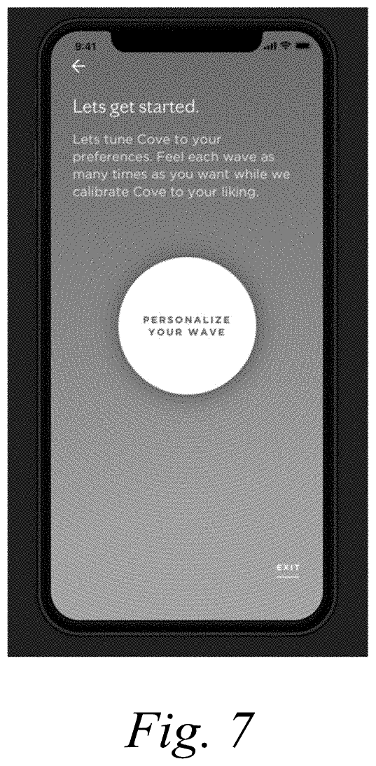

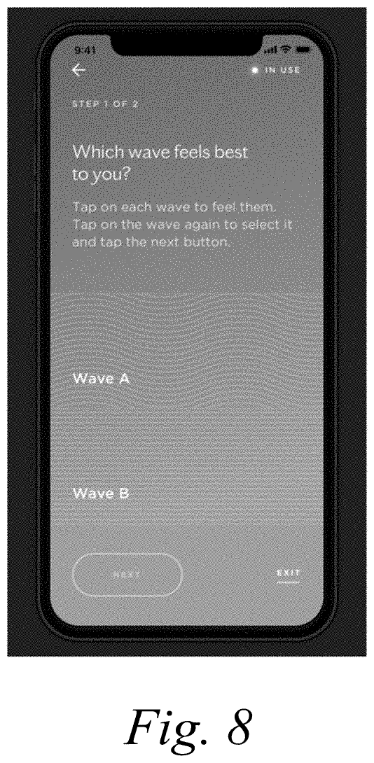

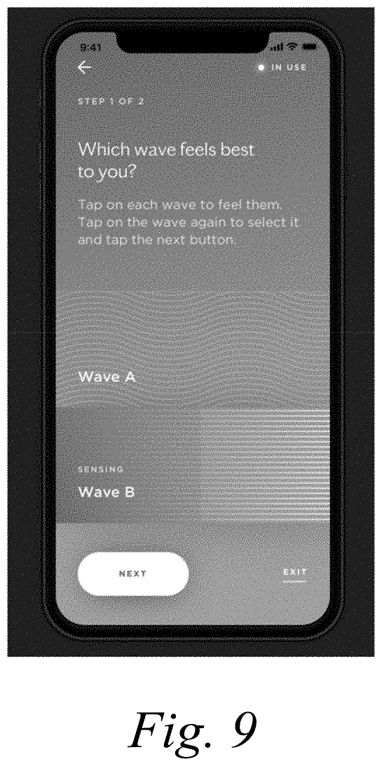

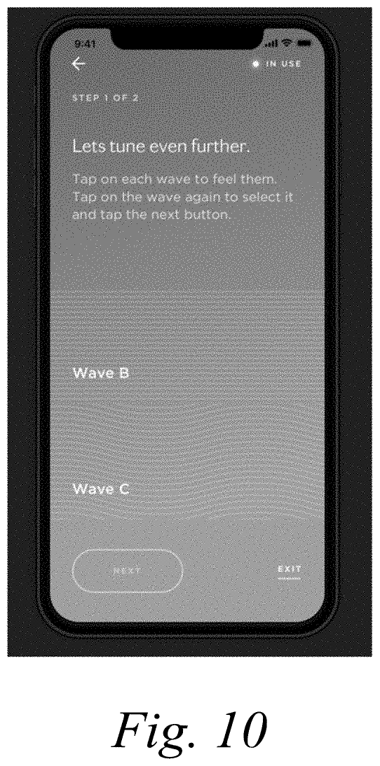

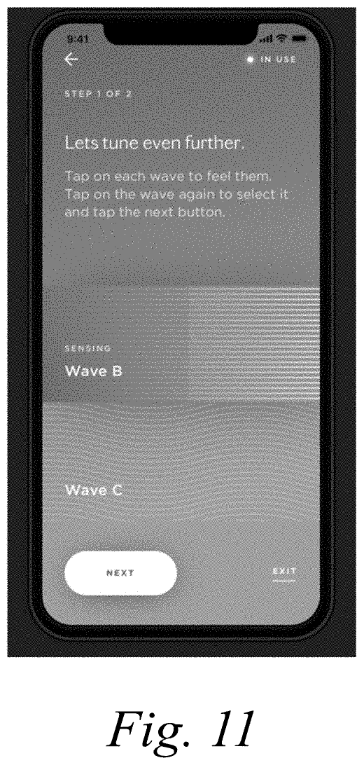

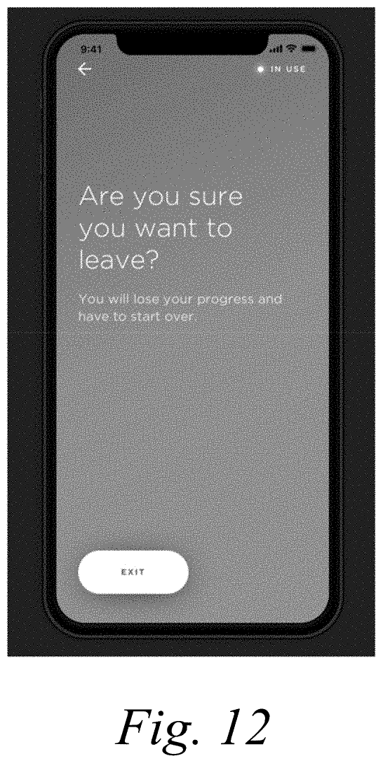

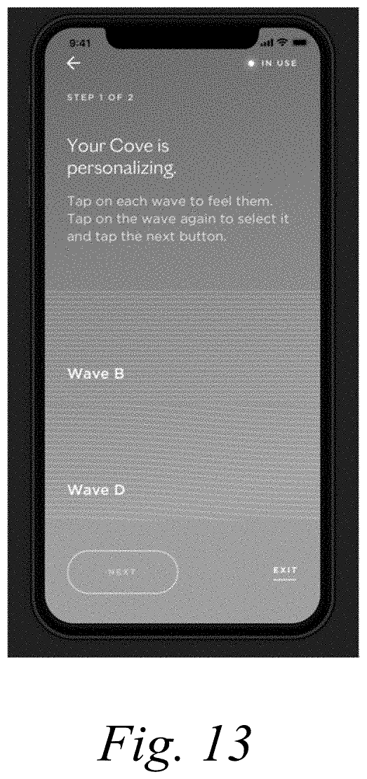

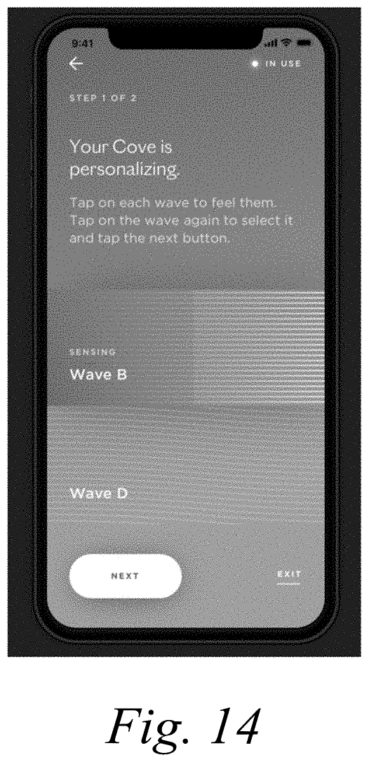

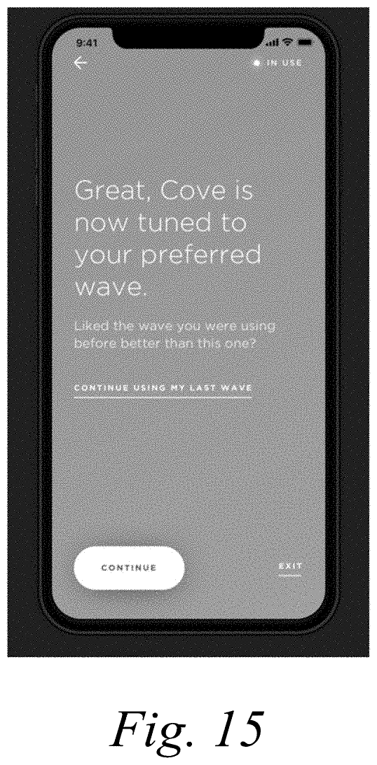

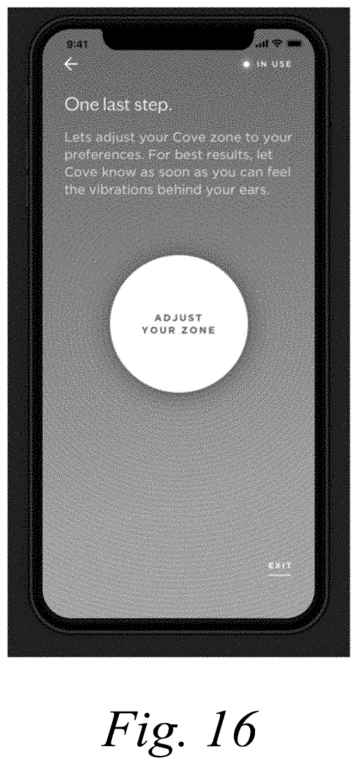

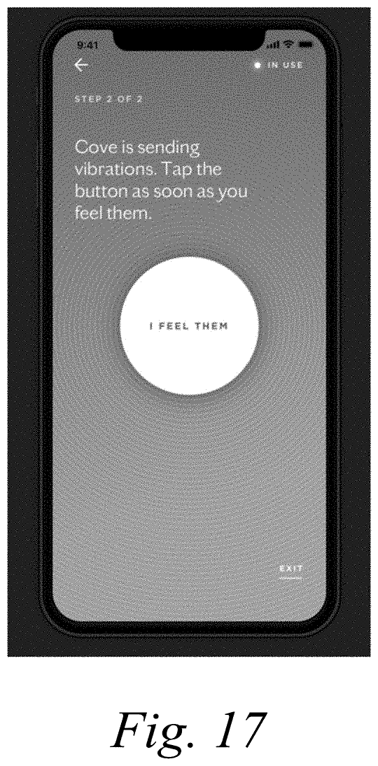

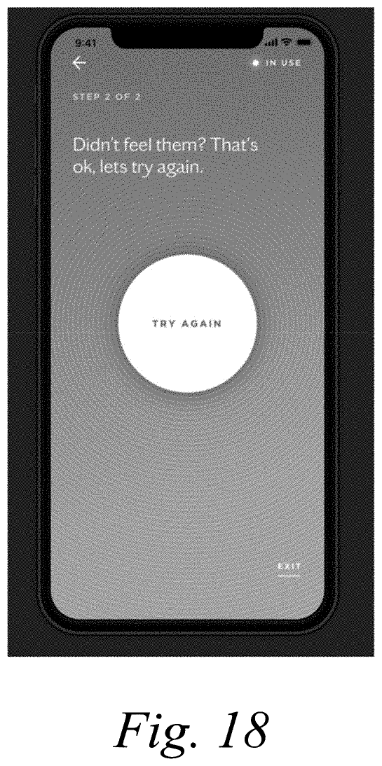

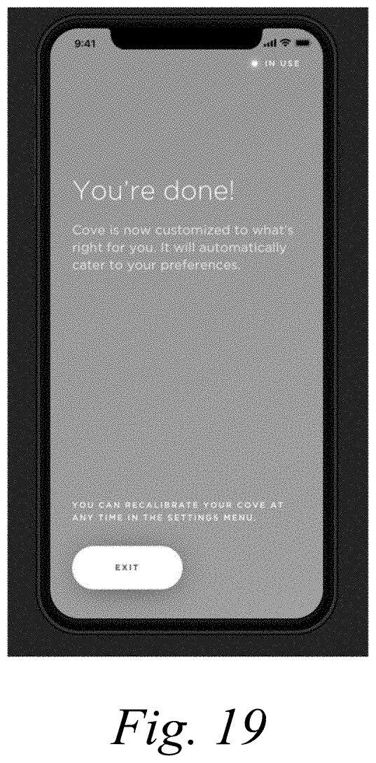

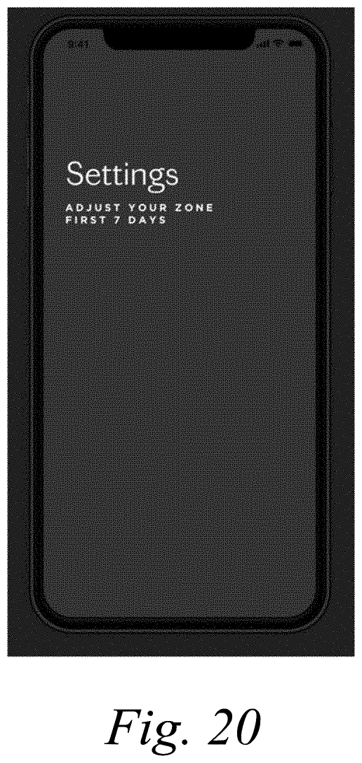

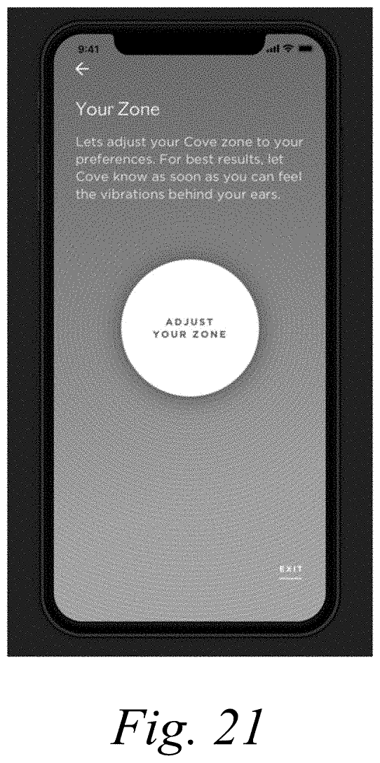

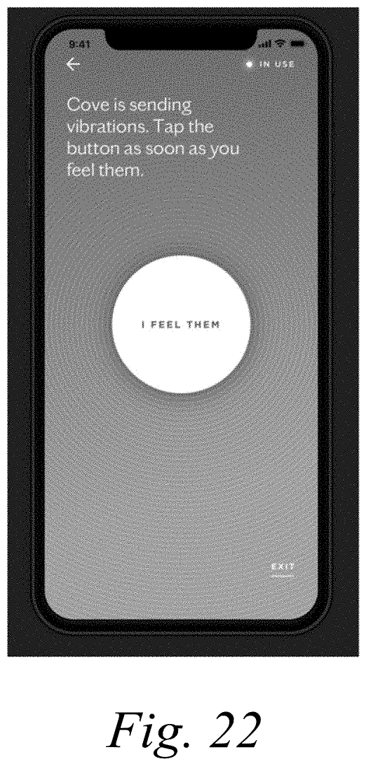

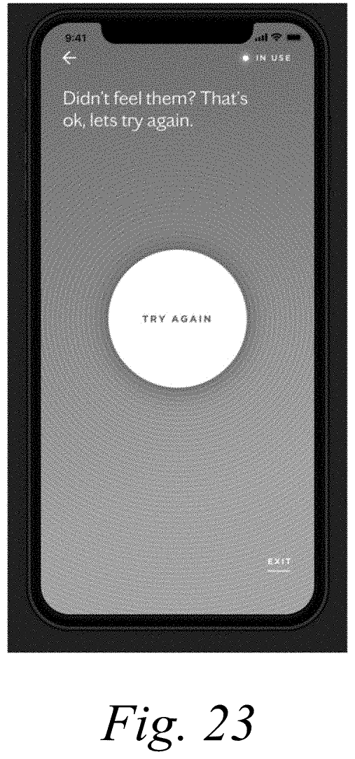

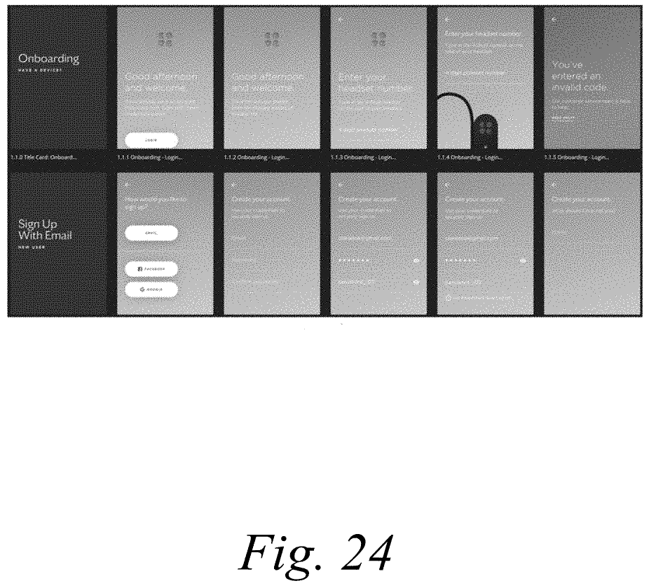

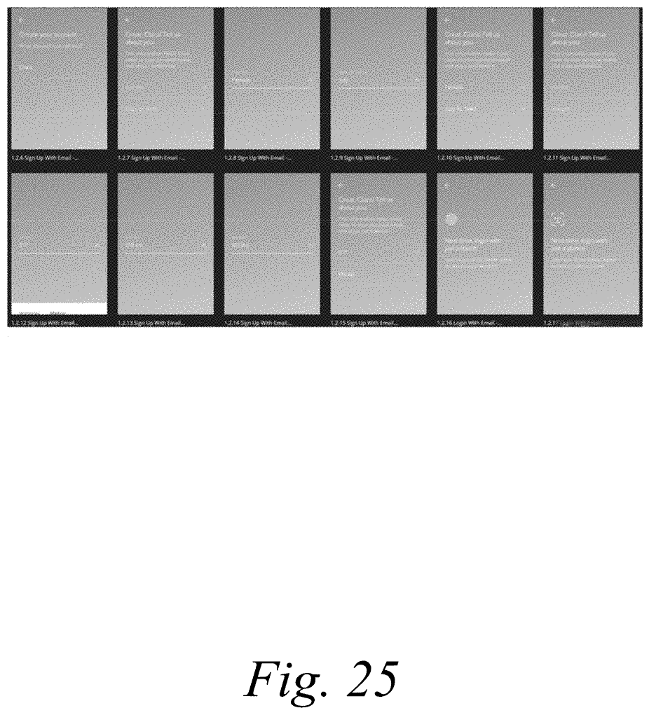

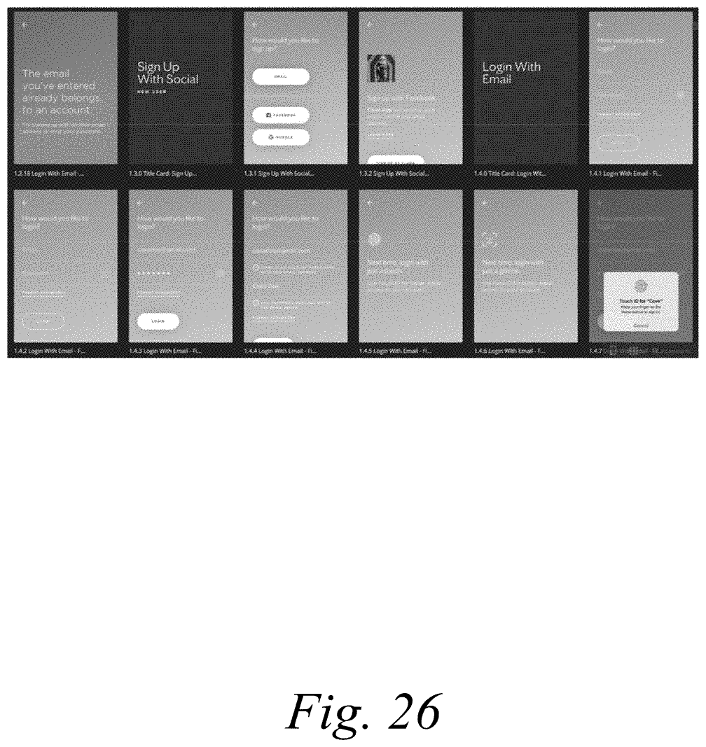

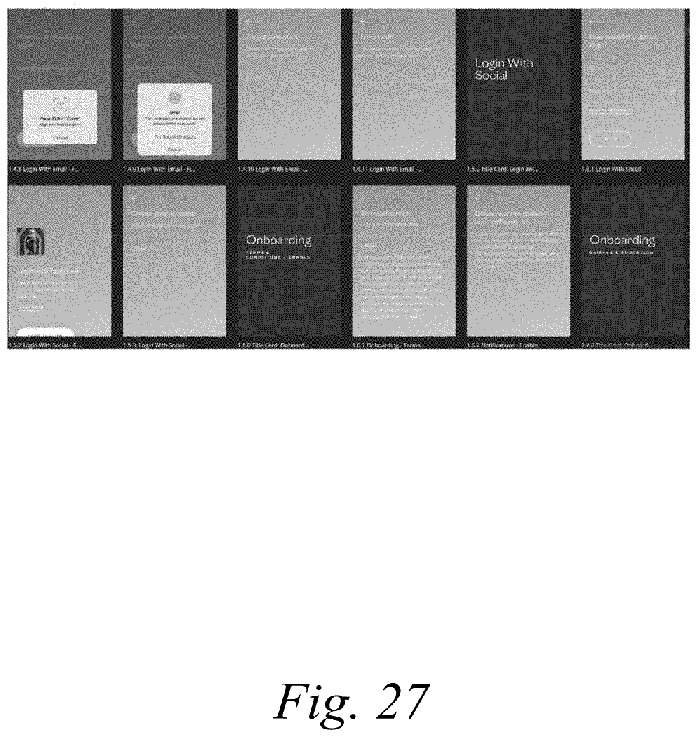

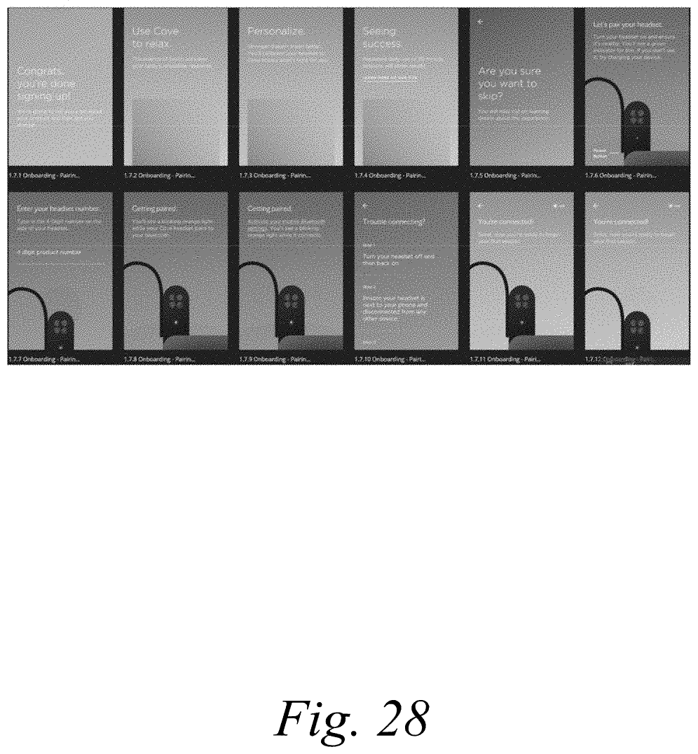

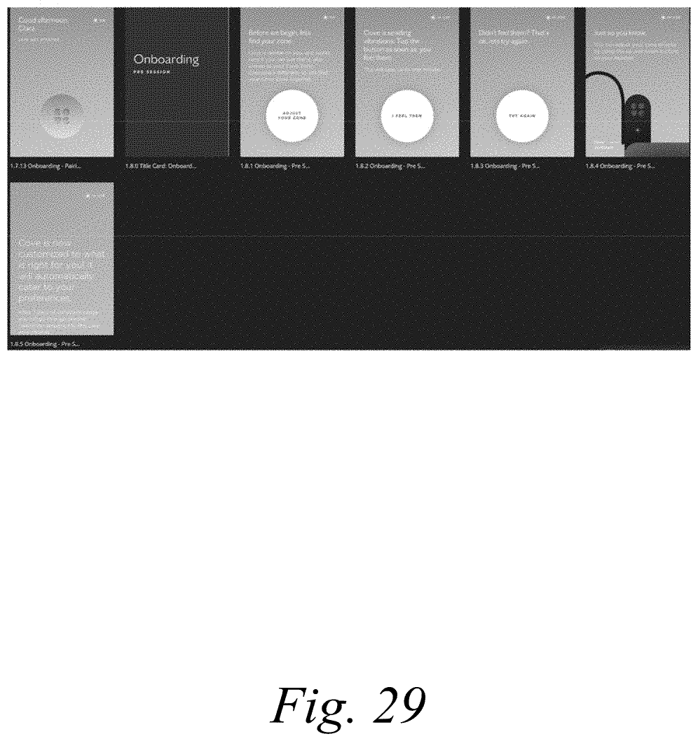

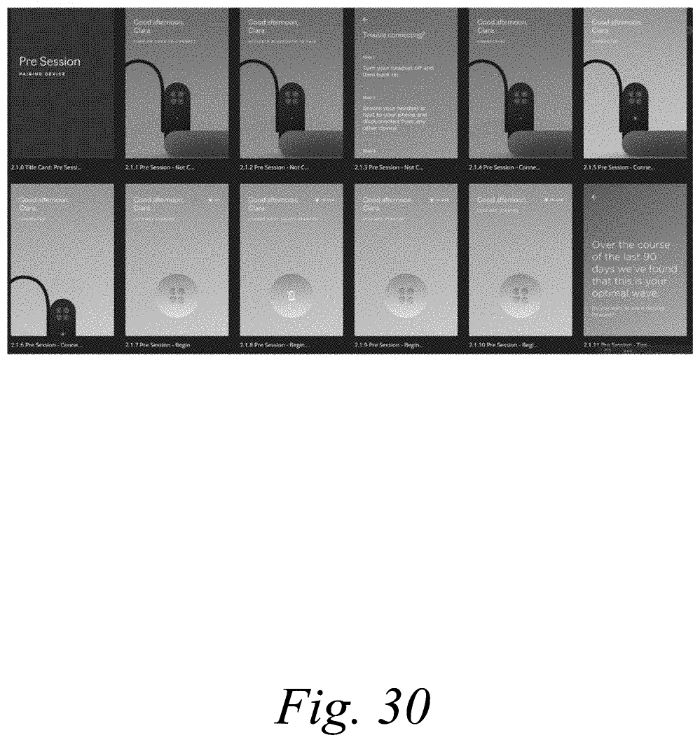

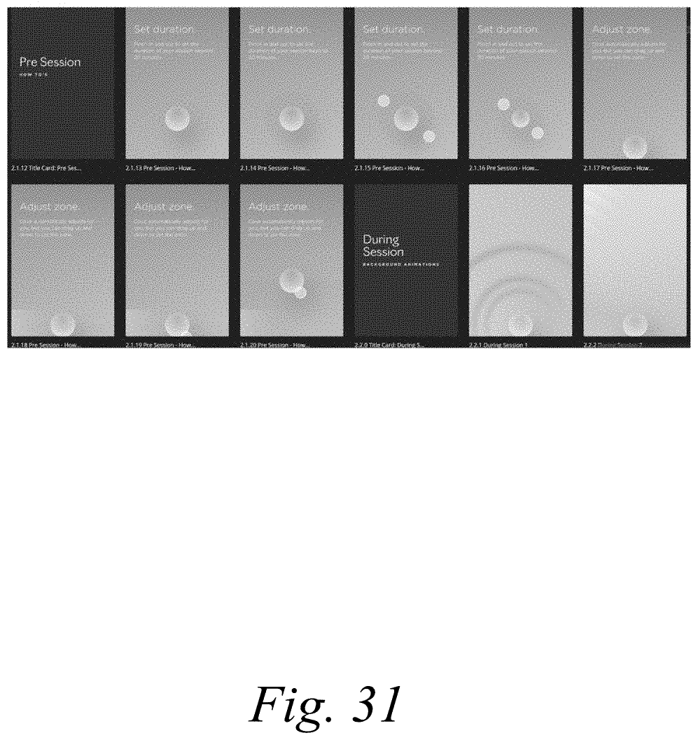

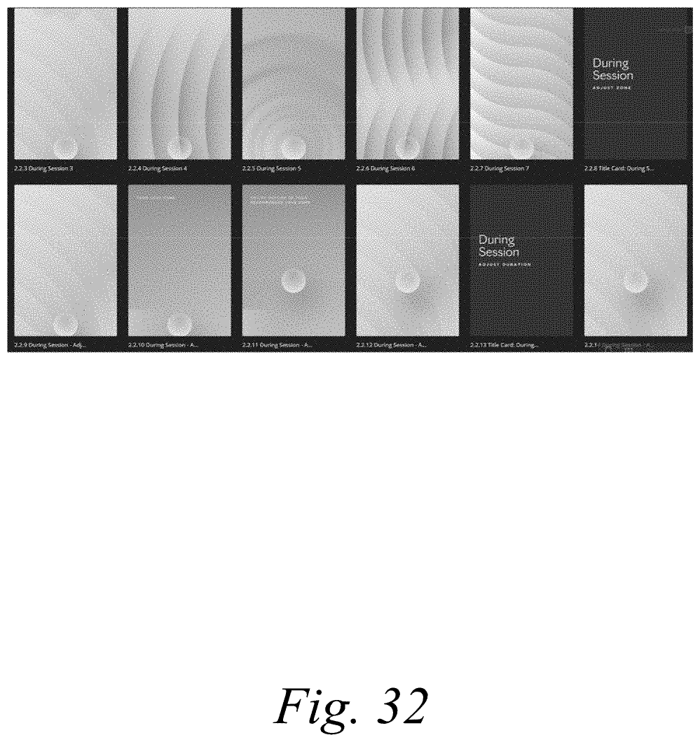

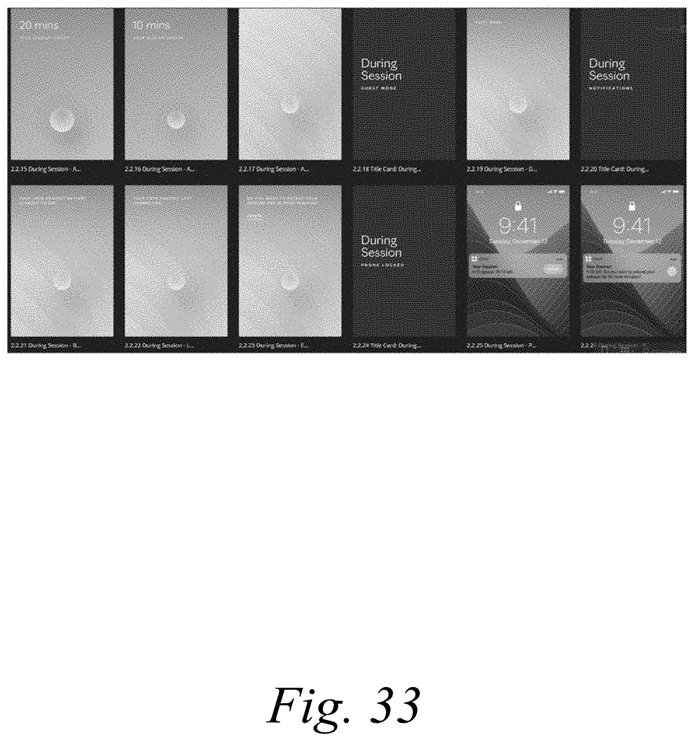

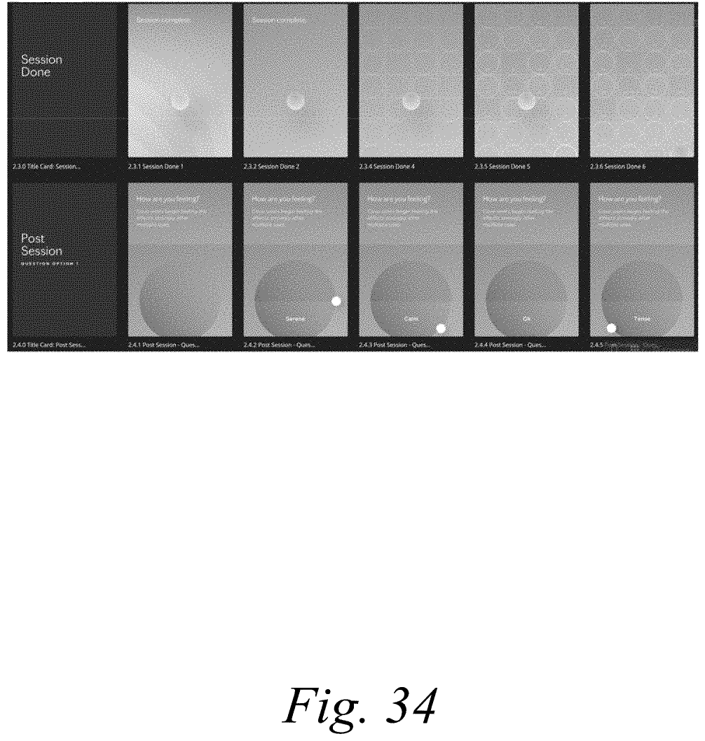

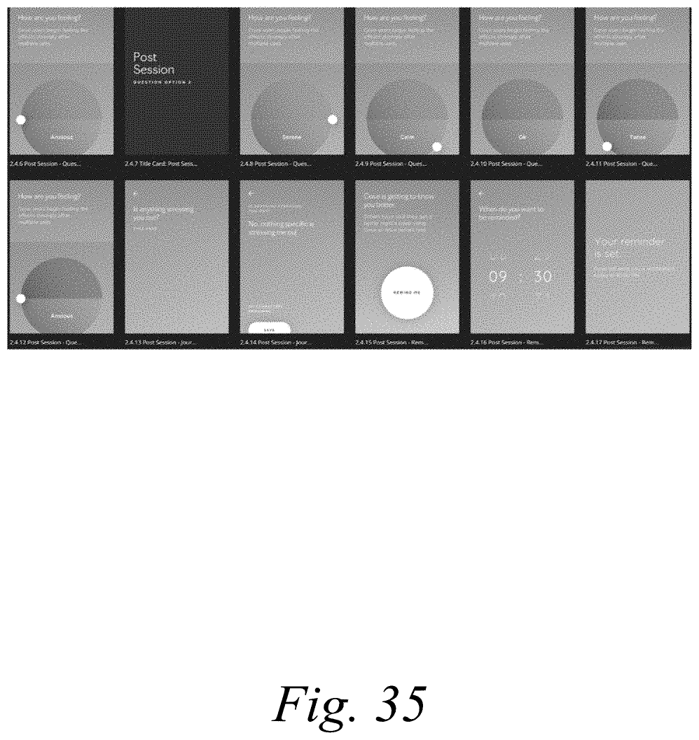

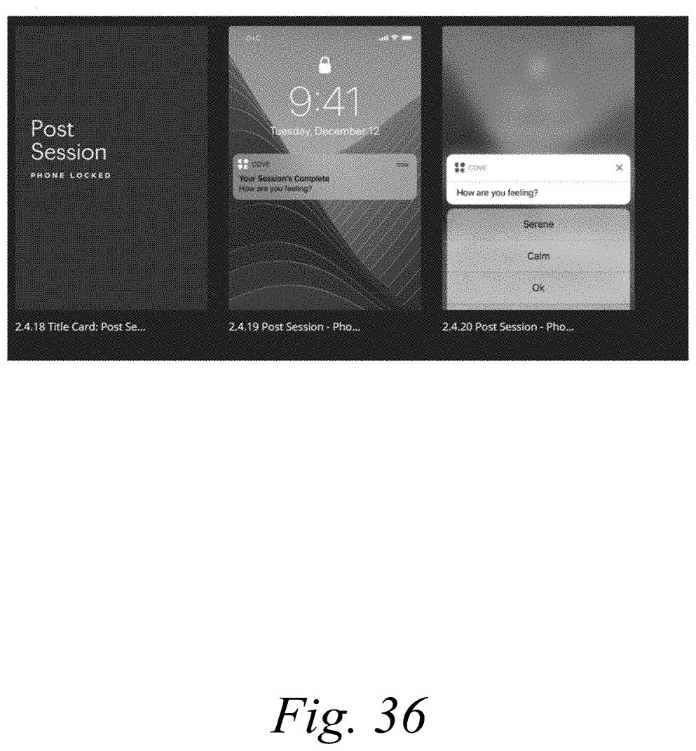

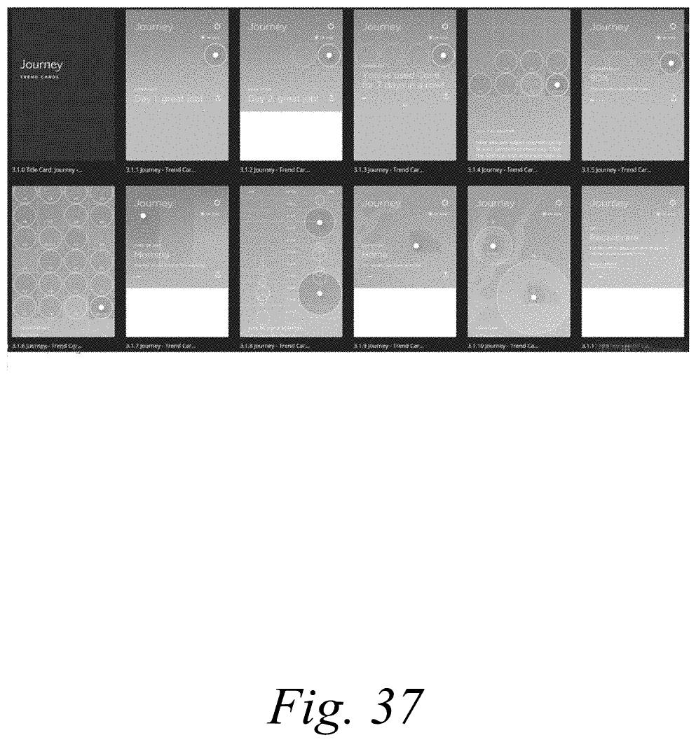

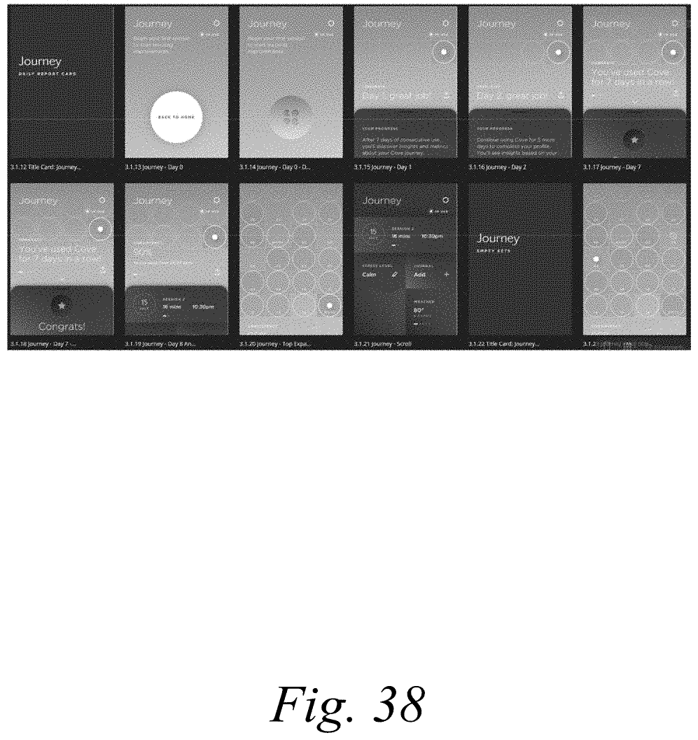

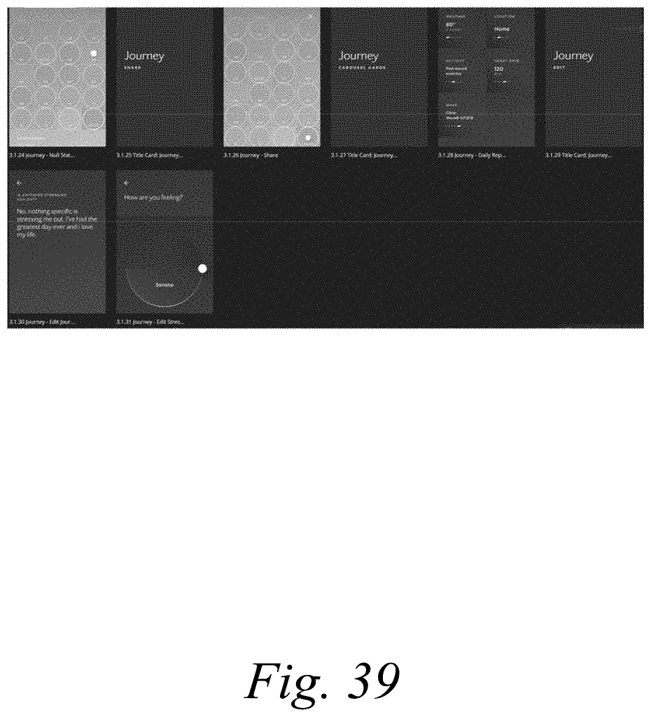

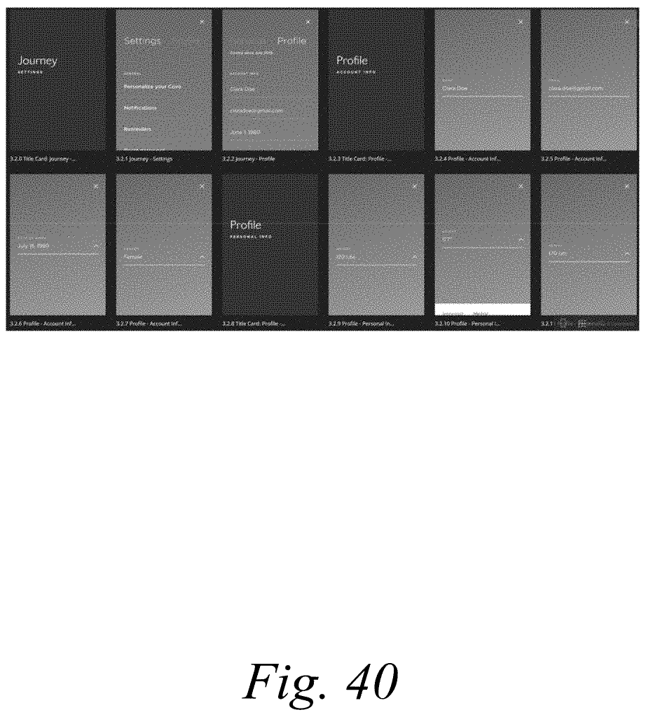

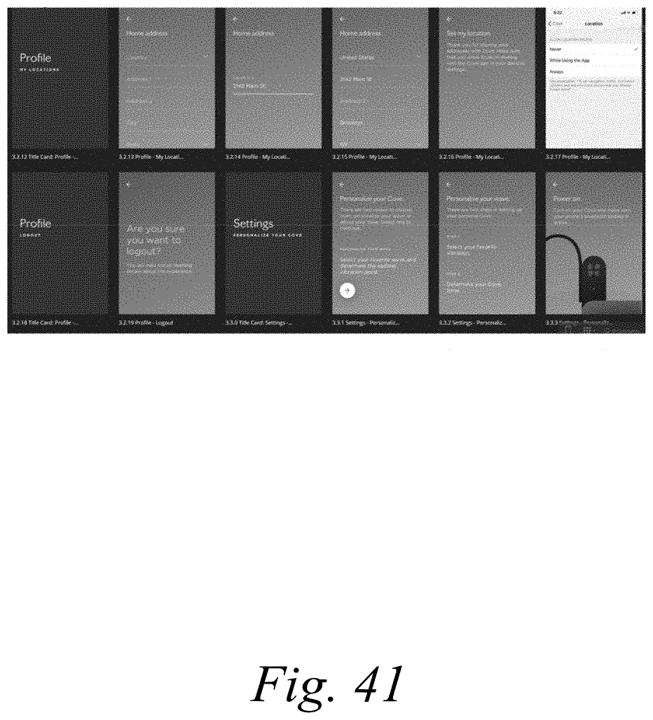

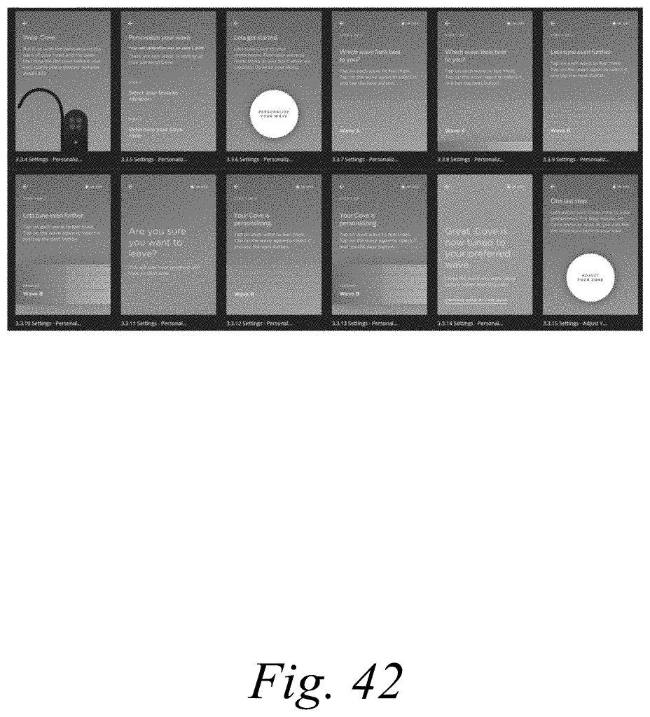

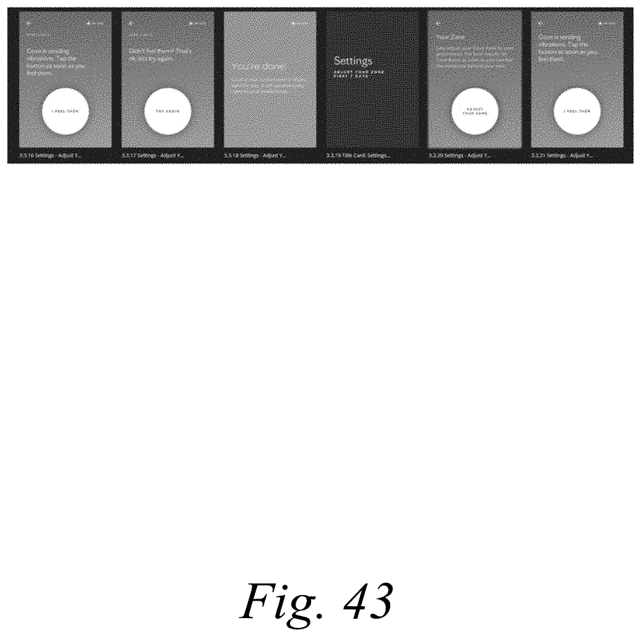

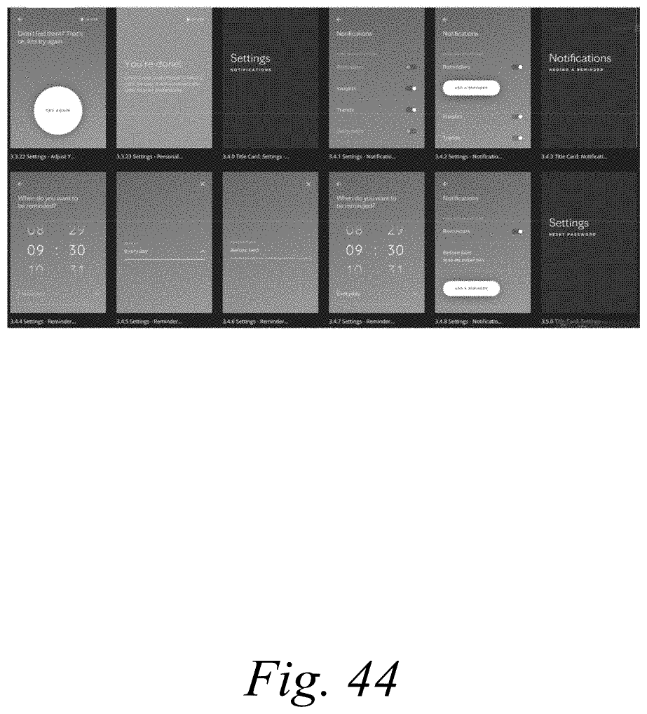

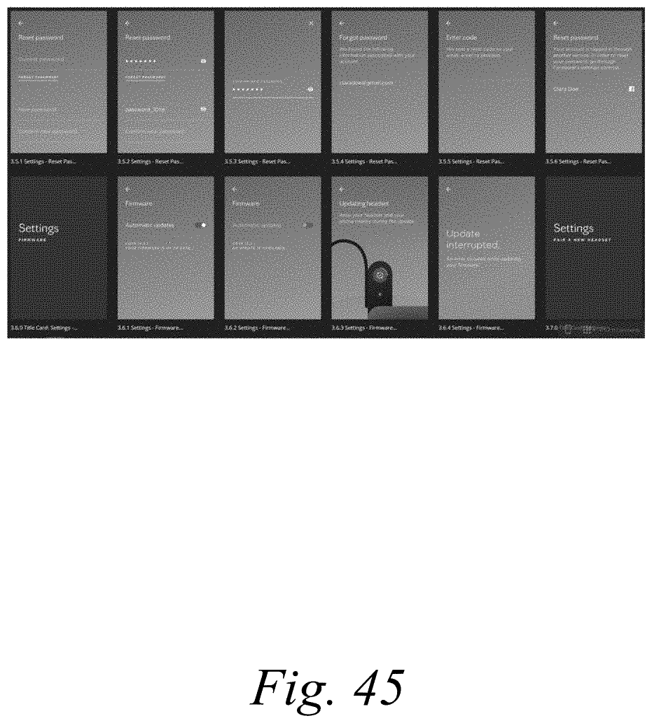

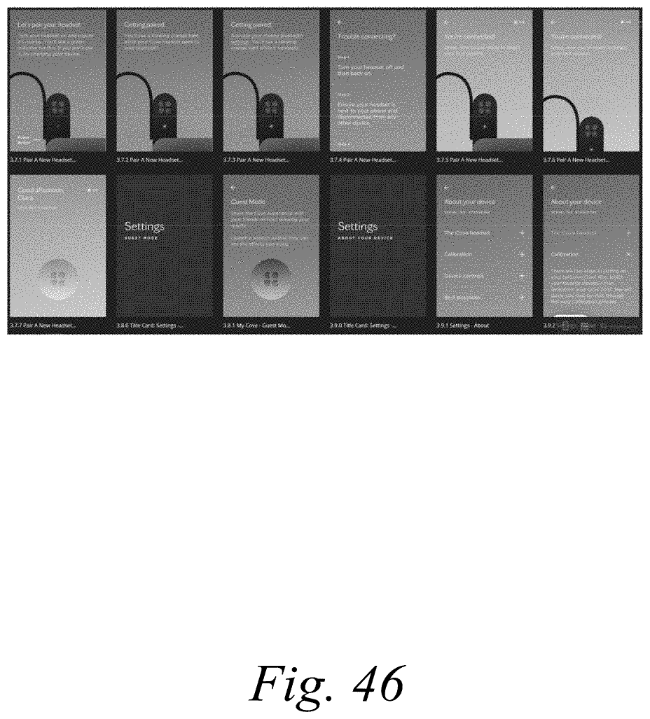

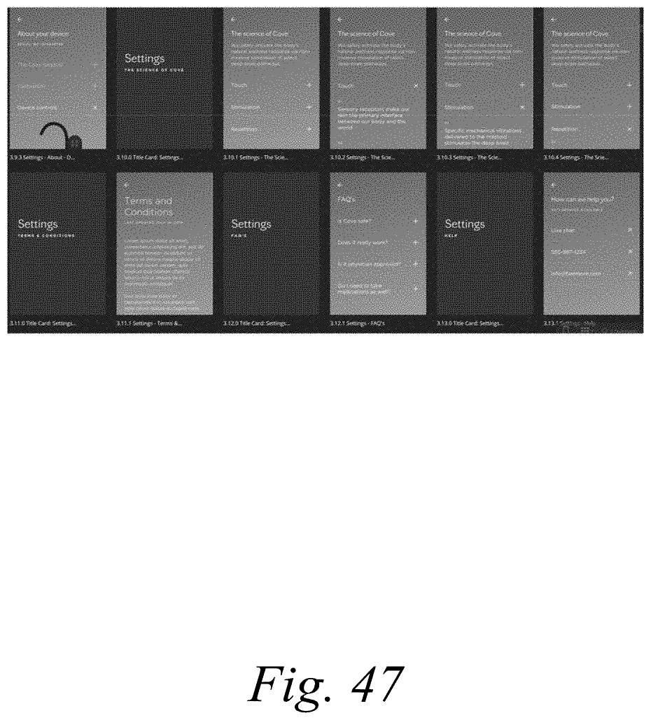

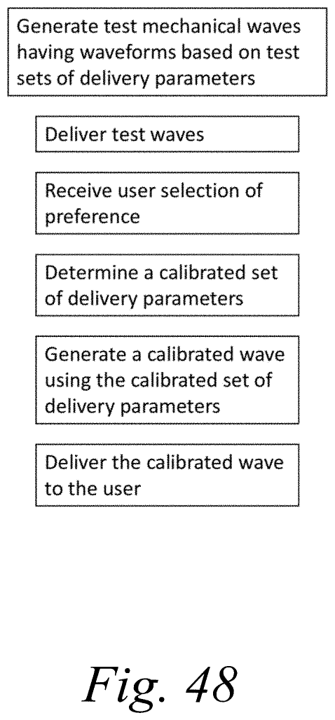

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DETAILED DESCRIPTION OF THE INVENTION

It is contemplated that systems, devices, methods, and processes of the claimed inventions described in this disclosure encompass variations and adaptations developed using information from the embodiments described herein. Adaptation and/or modification of the systems, architectures, devices, methods, and processes described herein may be performed, as contemplated by this description.

Throughout the description, where articles, devices, and systems are described as having, including, or comprising specific components, or where processes and methods are described as having, including, or comprising specific steps, it is contemplated that, additionally, there are articles, devices, and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously or at different times.

The mention herein of any publication, for example, in the Background section, is not an admission that the publication serves as prior art with respect to any of the claims presented herein. The Background section is presented for purposes of clarity and is not meant as a description of prior art with respect to any claim.

Documents are incorporated herein by reference as noted. Where there is any discrepancy in the meaning of a particular term, the meaning provided in the Definition section above is controlling.

Headers are provided for the convenience of the reader—the presence and/or placement of a header is not intended to limit the scope of the subject matter described herein.

As used herein, the terms “user” and “subject” are used interchangeably.

Signal development used to target C-tactile afferent nerves (mechanoreceptors) can be constrained by the endogenous response domain (the frequency(ies or the stimulation, the duration, the continuity, the strength, pressure and track over the skin, among others). The signals and effects described herein are designed to follow within the known response domains. In addition, these stimulation parameters, while specifically designed around the above characteristics, are also related to specific brain responses, in particular the range of frequencies associated with alpha waves, a range of brain waves associated with at least relaxation, calm focus and reduced anxiety and stress. Based on the overlap between the immediate localized skin response via CT afferents and the much more integrated brain responses as seen in changes in alpha brain waves, the approaches described herein utilize a differential calibration that allows individualization of the initial wave forms, based on subjective preference, which can be related to the differences in the specifics of an individuals preferred CT afferent response domain and the dominant frequency of the individual's alpha brain waves. In this way, the user can more closely couple the stimulation to their individual endogenous (or preferred) response domains.

In particular, when an individual user initially begins to use the devices, systems, and methods described herein, there is a period of calibration. In certain embodiments, the user is presented with stimulation at extremes (e.g., higher and lower) of the preferred response domains in the CT afferent and/or the individuals dominant alpha wave frequency. The user is asked to express (e.g., a choice on a digital application) a preference between the two signal frequencies. Given that feedback, the system will offer additional choices around that preferred frequency range, ask again for a differential preference and iterate the process to determine a final preferred initial stimulation frequency for the device. The process can be iterated, for example, after a period of continuous use, maybe 7 days or 30 days or in another process by individual choice as the user prefers.

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein.

Throughout the description, where apparatus and systems are described as having, including, or comprising specific components, or where processes and methods are described as having, including, or comprising specific steps, it is contemplated that, additionally, there are apparatus and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

In one embodiment, a method of providing mechanical stimulation to a user through a stimulation device is provided. The method comprises (a) providing a stimulation device containing a plurality of mechanical waves to a user, each mechanical wave having one or more vibrational waveforms; (b) delivering more than one of the plurality of mechanical waves to the user through the stimulation device to a location in proximity to a mastoid of the user; (c) receiving, by a processor of the stimulation device or a computing device in communication with the stimulation device, the user's selection of, or preference for, one or more of the plurality of mechanical waves that the user prefers; (d) modifying one or more of the plurality of mechanical waves provided to the user or selecting other mechanical waves to provide to the user, based, at least in part, on the user's selection of or preference for one or more of the plurality of mechanical waves that the user prefers; and (e) delivering the modified or other mechanical waves to the user through the stimulation device to a location in proximity to a mastoid of the user.

It should be appreciated that the methods, devices and systems of the present disclosure provide various benefits and advantages. As examples and not by way of limitation, the method, devices and systems of the present disclosure provide, among other things, a mechanical waves that are customized to the user to be delivered to the user in a way that provides the maximal benefit to the user, provides stimulation to a location in proximity to a mastoid of the user which provides the benefits and advantages described herein

In an aspect of at least one embodiment of the current disclosure, modifying the one or more plurality of mechanical waives provided to the user or selecting other mechanical waves to provide to the user is also based at least in part on the stimulation device or the user selecting and/or refining attributes of the mechanical waves over a set period of time and/or until a particular stop is reached by the user. In doing so, the user can select and modify the mechanical waves provided to the user to create a customized program and customized mechanical waves for each user based on their desires and the best impact. It should be appreciated that the components of the device described herein can be created using known components found in the marketplace.

In an aspect of at least one embodiment of the current disclosure, the user can modify the frequency, amplitude and/or the duration of the mechanical stimulation received by the user to customize exactly the stimulation they want to receive for the desired benefit and without exceeding their comfort and stimulation preferences without making the user uncomfortable or less likely to complete the program associated with the treatment provided by the devices disclosed herein.

In an aspect of at least one embodiment of the current disclosure, the user provides an indication that the user feels vibrations from the device and the device modifies one or more of the plurality of mechanical waves provided to the user or selects other mechanical waves to provide to the user based at least in part on the user's indication that the user feels vibrations from the device. By determining a baseline for where the user feels the vibrations from the device, the device can determine a range of parameters of the mechanical waves that will both be efficacious to help the user to reduce stress, improve sleep and provide other mental and physiological benefits and also not make the vibrations uncomfortable or undesirable to the user, which would prevent continued use and completion of the program. For example and not by way of limitation, by determining the baseline for where the user feels the vibrations, the device may determine that vibrations can decrease up to 75% and increase to 500% in frequency, amplitude and or duration of the mechanical stimulation based on the baseline determined by the user, which would be particular to each and ever user.

In an aspect of at least one embodiment of the current disclosure, the device receives from one or more devices one or more biofeedback signals of the user and determines and/or modifies the mechanical waves provided to the user through the stimulation device based on the received one or more biofeedback signals. As an example, and not by way of limitation, examples of the types of biofeedback includes but is not limited a fitness tracker (e.g., pedometer); a heart-rate monitor; e.g., an electrocardiograph (EKG) monitor; e.g., an electroencephalography (EEG) monitor; e.g., an accelerometer; e.g., a blood-pressure monitor; e.g., a galvanic skin response (GSR) monitor); one or more biofeedback signals (e.g., step count, hear-rate statistics, such as average heart rate, heart-rate variability, etc., EKG, EEG, blood pressure, GSR, etc.)

In an aspect of at least one embodiment of the current disclosure, the plurality of mechanical waves to provided to the user through the stimulation device stimulates both the vagal nerve of the user and the C-tactile afferents of the user. Having tested various nerves and portions of the body, it was determined that stimulating the vagal nerve and C-tactile afferents of the user provided the benefits and advantages of the present invention without providing negative effects to the user.

In an aspect of at least one embodiment of the current disclosure, the plurality of mechanical waves provided to the user through the stimulation device stimulates a combination of the vagal nerve of the user, the trigeminal nerve of the user and the C-tactile afferents of the user and increases the expression of a Piezo2 protein in the user. Having tested various nerves and portions of the body and their effect on protein expression on the user, it was determined that stimulating the vagal nerve, the trigeminal nerve, the C-tactile afferents of the user and increasing expression of Piezo2 protein in the user, provided the benefits and advantages of the present invention without providing negative effects to the user.

In an aspect of at least one embodiment of the current disclosure, delivering more than one of the plurality of mechanical waves to the user through the stimulation device also includes a haptic, audio, visual, or other feedback alert provided to the user through the stimulation device or a computing device in communication with the stimulation device indicating the beginning or ending of delivery of the mechanical wave. By doing so the user can more clearly and accurately determine the beginning and ending of specific waves provided to the user improving the efficacy and experience provided to the user

In another embodiment of the current disclosure, a transcutaneous neuromodulation device for providing transcutaneous mechanical stimulation to a user is provided. The device includes one or more mechanical transducers operable to produce mechanical waves in response to an electronic drive signal and positioned to deliver the mechanical waves to the user at one or more body locations in proximity to one or more mastoid locations of the user, a controller board in communication with the one or more mechanical transducers of the neuromodulation device. The mechanical waves have a vibrational waveform that provides transcutaneous mechanical stimulation to the user and the controller board comprises a controller board processor and controller board memory having instructions stored in the memory that when the instructions are executed on the neuromodulation device causes the electronic drive signal to generate a plurality of mechanical waves, each having a vibrational waveform based at least in part on parameters of the mechanical waves selected by the user. I providing this level of customization and stimulation to the indicated areas, a truly customizable program can be provided to the user.

In another embodiment of the current disclosure, a mechanical stimulation device is provided. The device contains two or more mechanical transducers operable to produce mechanical waves in response to an electronic drive signal and positioned to deliver the mechanical waves to the user at one or more body locations in proximity to one or more mastoid locations of the user, a controller board in communication with the one or more mechanical transducers of the mechanical stimulation device, a processor in communication with the controller board and memory in communication with the controller board. The components of said device are known to one of ordinary skill in the art and can be obtained from various sources. The mechanical transducers of the stimulation device provide a mechanical wave having a vibrational waveform that provides transcutaneous mechanical stimulation to the user in proximity to one or more mastoid locations of the user, which provides the user with maximum benefit and results with the lowest amount of side effects and discomfort.

While the invention has been particularly shown and described with reference to specific preferred embodiments, it should be understood by those skilled in the art that various changes in form and detail may be made therein without departing from the spirit and scope of the invention as defined by the appended claims.

What is claimed is:

1. A method of providing mechanical stimulation to a user through a stimulation device, the method comprising:
(a) providing a stimulation device containing a plurality of mechanical waves to a user, each mechanical wave having one or more vibrational waveforms;
(b) delivering more than one of the plurality of mechanical waves to the user through the stimulation device to a location in proximity to a mastoid of the user;
(c) receiving, by a processor of the stimulation device or a computing device in communication with the stimulation device, the user's selection of, or preference for, one or more of the plurality of mechanical waves that the user prefers;
(d) modifying one or more of the plurality of mechanical waves provided to the user or selecting other mechanical waves to provide to the user, based, at least in part, on the user's selection of or preference for one or more of the plurality of mechanical waves that the user prefers; and
(e) delivering the modified or other mechanical waves to the user through the stimulation device to a location in proximity to a mastoid of the user.

2. The method of claim 1, wherein modifying the one or more plurality of mechanical waives provided to the user or selecting other mechanical waves to provide to the user is also based at least in part on the stimulation device or the user selecting and/or refining attributes of the mechanical waves over a set period of time and/or until a particular stop is reached by the user.

3. The method of claim 2, wherein the attributes of the mechanical waves that are modified by the user comprise one or more members selected from the group consisting of the frequency of the vibrational waveform, the amplitude of the vibrational waveform, and/or the duration of the mechanical stimulation received by the user.

4. The method of claim 1, further comprising:
receiving an indication from the user that the user feels vibrations from the device; and
modifying one or more of the plurality of mechanical waves provided to the user or selecting other mechanical waves to provide to the user based at least in part on the user's indication that the user feels vibrations from the device.

5. The method of claim 1, further comprising:
receiving from one or more devices one or more biofeedback signals of the user; and
determining and/or modifying the mechanical waves provided to the user through the stimulation device based on the received one or more biofeedback signals.

6. The method of claim, 1 wherein delivering more than one of the plurality of mechanical waves to the user through the stimulation device stimulates both the vagal nerve of the user and the C-tactile afferents of the user.

7. The method of claim 1, wherein delivering more than one of the plurality of mechanical waves to the user through the stimulation device stimulates one or more members elected from the group consisting of: a vagal nerve of the user; a trigeminal nerve of the user; a C-tactile afferents of the user and/or increasing the expression of a Piezo2 protein in the user.

8. The method of claim 1, wherein delivering more than one of the plurality of mechanical waves to the user through the stimulation device also includes a haptic, audio, visual, or other feedback alert provided to the user through the stimulation device or a computing device in communication with the stimulation device indicating the beginning or ending of delivery of each mechanical wave.

9. A transcutaneous neuromodulation device for providing transcutaneous mechanical stimulation to a user, the device comprising:
one or more mechanical transducers operable to produce mechanical waves in response to an electronic drive signal and positioned to deliver the mechanical waves to the user at one or more body locations in proximity to one or more mastoid locations of the user; and
a controller board in communication with the one or more mechanical transducers of the neuromodulation device;
wherein the mechanical waves having vibrational waveforms that provides transcutaneous mechanical stimulation to the user; and
wherein the controller board comprises a controller board processor and controller board memory having instructions stored in the memory that when the instructions are executed on the neuromodulation device causes the electronic drive signal to generate a plurality of mechanical waves, each having a vibrational waveform based at least in part on parameters of the mechanical waves selected by the user.

10. The neuromodulation device of claim 9, wherein the user may select and/or refine attributes of the mechanical waves of the device over a set period of time using the device or a computing device connected to neuromodulation device.

11. The neuromodulation device of claim 9, wherein the neuromodulation device or the user may modify the attributes of the mechanical waves that comprise one or more members selected from the group consisting of the frequency of the vibrational waveform, the amplitude of the vibrational waveform, and/or the duration of the mechanical stimulation received by the user.

12. The neuromodulation device of claim 9, wherein the device is capable of receiving and processing an indication from the user that the user feels vibrations from the device and modifies one or more of the plurality of mechanical waves provided to the user or selects other mechanical waves to provide to the user based at least in part on the user's indication that they feel vibration from the device.

13. The neuromodulation device of claim 9, wherein the device is capable of receiving from one or more devices, one or more biofeedback signals of the user and selects different mechanical waves and/or modifies the mechanical waves provided to the user through the neurmodulation device based on the received one or more biofeedback signals of the user.

14. The neuromodulation device of claim 9, wherein the plurality of mechanical waves produced by the neuromodulation devices and provided to the user stimulates both the vagal nerve of the user and the C-tactile afferents of the user.

15. The neuromodulation device of claim 9, wherein the plurality of mechanical waves provided to the user through the device stimulates one or more members elected from the group consisting of: a vagal nerve of the user; a trigeminal nerve of the user; a C-tactile afferents of the user and/or increases the expression of a Piezo2 protein in the user.

16. The neuromodulation device of claim 9, wherein the device also provides a haptic, audio, visual, or other feedback alert provided to the user through the stimulation device or a computing device in communication with the stimulation device indicating to the user the beginning or ending of delivery of each mechanical wave.

17. A mechanical stimulation device, the device comprising:
two or more mechanical transducers operable to produce mechanical waves in response to an electronic drive signal and positioned to deliver the mechanical waves to the user at one or more body locations in proximity to one or more mastoid locations of the user; and
a controller board in communication with the one or more mechanical transducers of the mechanical stimulation device;
a processor in communication with the controller board; and
memory in communication with the controller board;
wherein the mechanical transducers of the stimulation device provide mechanical waves having a vibrational waveform that provides transcutaneous mechanical stimulation to the user in proximity to one or more mastoid locations of the user.
The neuromodulation device of claim 9, wherein the plurality of mechanical waves produced by the neuromodulation devices and provided to the user stimulates both the vagal nerve of the user and the C-tactile afferents of the user.

18. The mechanical stimulation device of claim 17, wherein the device stimulates one or more members elected from the group consisting of: a vagal nerve of the user; a trigeminal nerve of the user; a C-tactile afferents of the user and/or increases the expression of a Piezo2 protein in the user.

19. The mechanical stimulation device of claim 17, wherein the plurality of mechanical waves produced by the devices and provided to the user stimulates both the vagal nerve of the user and the C-tactile afferents of the user.

20. The mechanical stimulation device of claim 17, wherein the device also provides a haptic, audio, visual, or other feedback alert provided to the user through the stimulation device or a computing device in communication with the stimulation device indicating to the user the beginning or ending of delivery of each mechanical wave.
